(19) United States
(12) Patent Application Publication
(10) Pub. No.: US 2025/0064132 A1
(43) Pub. Date: Feb. 27, 2025

(54) VENTILATION STRUCTURE AND AEROSOL GENERATING APPARATUS

(71) Applicant: SHENZHEN WOODY VAPES TECHNOLOGY CO., LTD., Shenzhen (CN)

(72) Inventors: Yongwen PENG, Shenzhen (CN); Youming LIU, Shenzhen (CN); Yanming NIU, Shenzhen (CN)

(73) Assignee: SHENZHEN WOODY VAPES TECHNOLOGY CO., LTD., Shenzhen (CN)

(21) Appl. No.: 18/810,494

(22) Filed: Aug. 20, 2024

(30) Foreign Application Priority Data
Aug. 21, 2023 (CN) 202311053716.3

(51) Int. Cl. A24F 40/48; A24F 40/10; A24F 40/42

(52) U.S. Cl. CPC A24F 40/48; A24F 40/42; A24F 40/10

(57) ABSTRACT

A ventilation structure and an aerosol generating apparatus are provided. The ventilation structure includes a liquid storage tank, an atomization chamber located on one side of the liquid storage tank, and a supporting frame fixedly connected to the liquid storage tank. The supporting frame includes a ventilation channel in communication with the liquid storage tank, the installation groove and the atomization chamber, and an installation groove. The ventilation channel is gradually increased in diameter from an end in communication with the installation groove toward the liquid storage tank. The ventilation channel is of a structure with a bigger top and a smaller bottom, so that the aerosol generating substrate leaking into the ventilation channel can be rapidly guided into the installation groove and stored in the installation groove.

### CROSS-REFERENCE TO RELATED APPLICATION

This application claims the priority to Chinese Application No. 202311053716.3, filed on Aug. 21, 2023, the contents of which are incorporated herein by reference in their entirety.

### TECHNICAL FIELD

The present disclosure relates to the field of electronic product manufacturing, and in particular, to a ventilation structure and an aerosol generating apparatus.

### BACKGROUND

As electronic products become popular, the demand for aerosol generating apparatuses is increasing. Currently, a commercial aerosol generating apparatus mainly includes a liquid storage assembly for storing an aerosol generating substrate, and an atomization assembly for atomizing an aerosol generating substrate to generate an aerosol. In a case where air pressure inside a liquid storage tank is low, air enters the liquid storage tank from an atomization chamber through a ventilation channel, so that a balance between the air pressure inside the liquid storage tank and the air pressure inside the atomization chamber is achieved, thereby the liquid is supplied smoothly. To solve the problem that a part of the aerosol generating substrate flows with air and leaks out of the aerosol generating apparatus, a labyrinth structure or a Tesla valve is commonly used for the ventilation channel of the currently commercial aerosol generating apparatus. However, a part of the aerosol generating substrate will be accumulated in the ventilation channel in the course of air entering the liquid storage tank from the atomization chamber through the ventilation channel. Thus, before ventilation, the aerosol generating substrate accumulated in the ventilation channel is required to be pushed back to the liquid storage tank by air, so that the ventilation efficiency of the aerosol generating apparatus is reduced.

### SUMMARY

Embodiments of the present disclosure provide a ventilation structure and an aerosol generating apparatus to prevent leakage of aerosol generating substrate and improve ventilation efficiency.

The present disclosure provides a ventilation structure, including:
a liquid storage tank;
an atomization chamber located on one side of the liquid storage tank; and
a supporting frame fixedly connected to the liquid storage tank, a ventilation channel and an installation groove being formed in the supporting frame, the liquid storage tank being in communication with the installation groove and the atomization chamber through the ventilation channel, and the ventilation channel being gradually increased in diameter from an end in communication with the installation groove toward the liquid storage tank.

According to the ventilation structure provided in the present disclosure, the ventilation channel includes a first channel and a second channel, the liquid storage tank is in communication with the installation groove through the first channel, the first channel is in communication with the atomization chamber through the second channel.

According to the ventilation structure provided in the present disclosure, the second channel includes a first connecting section, a bending section, and a second connecting section, one end of the first connecting section is in communication with the first channel, the first connecting section extends from an end in communication with the first channel along a direction close to the liquid storage tank and away from the first channel to be connected with the bending section, another end of the bending section is connected with the second connecting section, and another end of the second connecting section is in communication with the atomization chamber.

According to the ventilation structure provided in the present disclosure, an angle between the first connecting section and the first channel in a direction toward the liquid storage tank is greater than 30 degrees.

According to the ventilation structure provided in the present disclosure, an outer wall of the supporting frame is recessed inwardly to form the ventilation channel, and the ventilation channel does not penetrate through the supporting frame.

According to the ventilation structure provided in the present disclosure, the outer wall of the supporting frame is recessed inwardly to form the installation groove, and both ends of the installation groove extend through both sides of the supporting frame in a direction perpendicular to the first channel.

According to the ventilation structure provided in the present disclosure, a depth of the installation groove recessed is greater than a depth of the ventilation channel recessed.

According to the ventilation structure provided in the present disclosure, the ventilation channel includes one first channel and two second channels which are respectively located on both sides of the first channel.

According to the ventilation structure provided in the present disclosure, the ventilation structure further includes an absorber, which is arranged in the installation groove and configured for storing the aerosol generating substrate.

The present disclosure also provides an aerosol generating apparatus including:
a ventilation structure, which is the ventilation structure according to any one of the above items.

The present disclosure provides a ventilation structure and an aerosol generating apparatus. The ventilation structure includes a liquid storage tank, an atomization chamber and a supporting frame. The atomization chamber is located on one side of the liquid storage tank. The supporting frame is fixedly connected to the liquid storage tank, and a ventilation channel and an installation groove are formed in the supporting frame. The ventilation channel is in communication with the liquid storage tank, the installation groove and the atomization chamber. The ventilation channel is gradually increased in diameter toward the liquid storage tank from an end in communication with the installation groove. According to the present disclosure, due to the fact that the diameter of the ventilation channel is gradually increased from the end in communication with the installation groove toward the liquid storage tank (that is, the ventilation channel is of a structure with a bigger top and a smaller bottom), the leakage of the aerosol generating substrate into the ventilation channel can be rapidly guided into the installation groove, so that the aerosol generating substrate is stored in the installation groove. Thus, accumulation of the aerosol generating substrate in the ventilation channel and negative impact on the smooth operation of the aerosol generating apparatus during ventilation can be avoided, the leakage of the aerosol generating substrate into a mainboard to damage the aerosol generating apparatus is prevented, the ventilation efficiency of the aerosol generating apparatus is improved, and a defective product with a burnt core is avoided.

### BRIEF DESCRIPTION OF THE DRAWINGS

In order to clearly describe the technical solution of the embodiments of the present disclosure, references will now be made briefly to the accompanying drawings required for the description of the embodiments. It will be obvious that the accompanying drawings in the following description illustrate some embodiments of the present disclosure and other figures may be obtained to those skilled in the art without involving any inventive effort.

FIG. 1 is a structural diagram of a supporting frame according to an embodiment of the present disclosure;

FIG. 2 is a front view of a supporting frame according to an embodiment of the present disclosure;

FIG. 3 is a side view of a supporting frame according to an embodiment of the present disclosure;

FIG. 4 is a structural diagram of a supporting frame in combination with an absorber according to an embodiment of the present disclosure;

FIG. 5a to FIG. 5c are diagrams indicating a flow direction of air in the supporting frame according to an embodiment of the present disclosure;

FIG. 6 is a diagram indicating a flow direction of the aerosol generating substrate in the ventilation channel according to an embodiment of the present disclosure;

FIG. 7 is a cross-sectional view of an aerosol generating apparatus according to an embodiment of the present disclosure; and

FIG. 8 is an exploded view of an aerosol generating apparatus according to an embodiment of the present disclosure.

Each reference numeral in the figures is denoted by:

100: liquid storage tank; 200: atomization chamber; 300: supporting frame; 310: ventilation channel; 311: first channel; 312: second channel; 3121: first connecting section; 3122: bending section; 3123: second connecting section; 320: installation groove; 331: left side wall; 332: right side wall; 333: front wall; 334: rear wall; and 400: absorber.

### DETAILED DESCRIPTION

It should be noted that embodiments in the present disclosure and features in the embodiments may be combined with each other in a case where there is no conflict. A preferred embodiment of the present disclosure will now be described in detail with reference to the accompanying drawings.

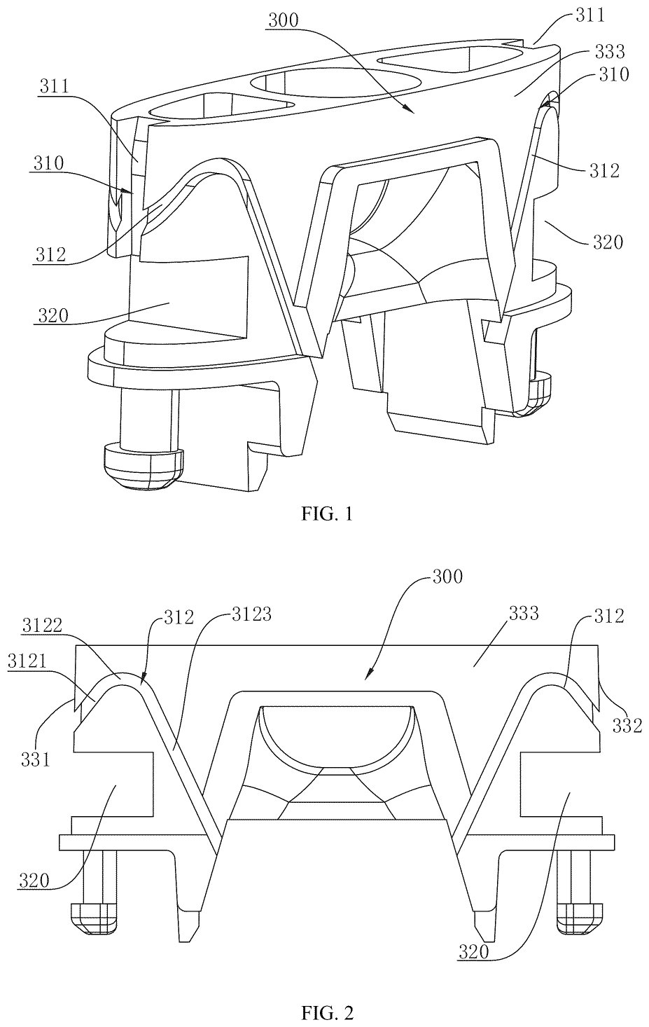

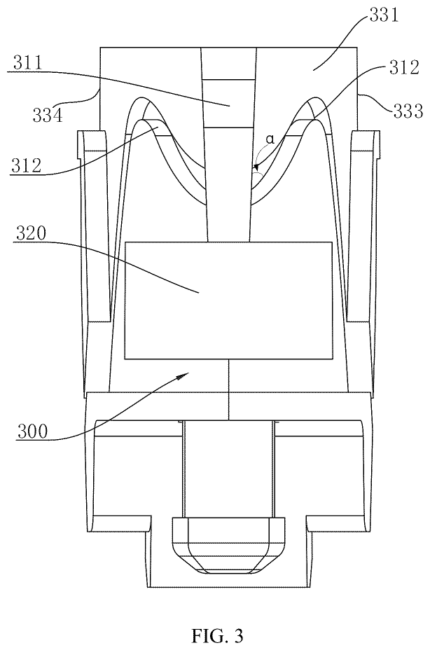

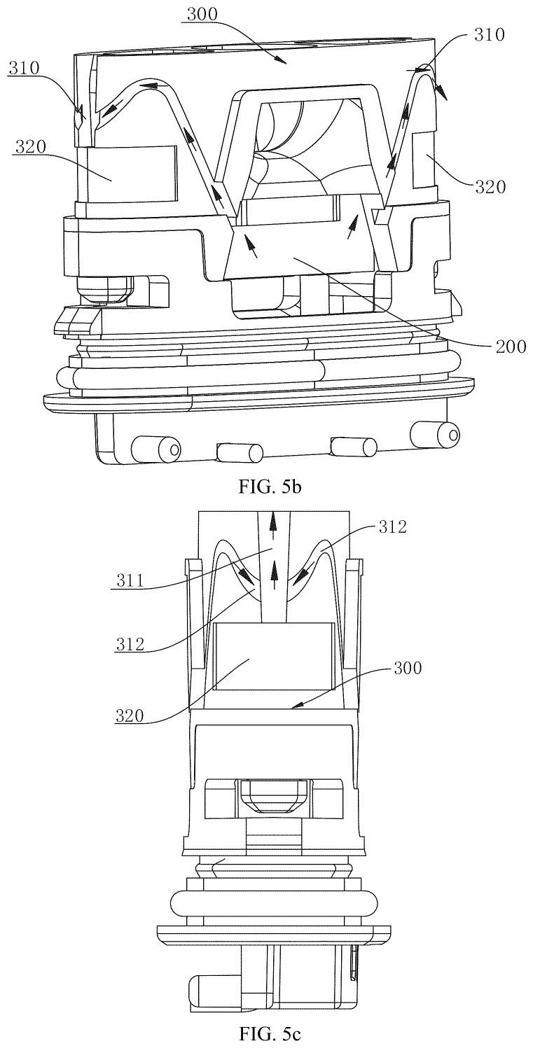

Referring to FIGS. 1 to 8, a ventilation structure according to an embodiment of the present disclosure is shown. The ventilation structure 1 includes a liquid storage tank 100, an atomization chamber 200 located on one side of the liquid storage tank 100, and a supporting frame 300 fixedly connected to the liquid storage tank. A ventilation channel 310 and an installation groove 320 are formed in the supporting frame 300. Through the ventilation channel 310, the liquid storage tank 100 is in communication with the installation groove 320 and the atomization chamber 200. The ventilation channel 310 is gradually increased in diameter toward the liquid storage tank 100 from its end in communication with the installation groove 320.

Specifically, the liquid storage tank 100 is configured to store an aerosol generating substrate. An atomization core is arranged in the atomization chamber 200. The atomization chamber 200 is arranged on one side of the liquid storage tank 100. The aerosol generating substrate flows from the liquid storage tank 100 to the atomization chamber 200 for atomization reaction in the atomization core, thereby generating an aerosol. The supporting frame 300 is made of ceramic. The supporting frame 300 is fixedly connected to the liquid storage tank 100. The atomization chamber 200 is located on another side, opposite to the side connected with the liquid storage tank 100, of the supporting frame 300. As such, the liquid storage tank 100 is located above the supporting frame 300, and the supporting frame 300 is located above the atomization chamber 200. A ventilation channel 310 and an installation groove 320 are formed in the supporting frame 300. The ventilation channel 310 is configured for the flow of air, and through the ventilation channel 310, the liquid storage tank 100 is in communication with the installation groove 320 and the atomization chamber 200. The ventilation channel 310 is gradually increased in diameter from an end in communication with the installation groove 320 toward an end in communication with the liquid storage tank 100. The diameter of the ventilation channel 310 refers to the sum of the distances from a center of the ventilation channel 310 to two opposite points on the periphery of the ventilation channel 310.

Therefore, during the ventilation process of the aerosol generating apparatus, air flows from the atomization chamber 200 to the liquid storage tank 100 through the ventilation channel 310, so that a balance between the air pressure in the atomization chamber 200 and the air pressure in the liquid storage tank 100 is achieved, the normal use of the aerosol generating apparatus is ensured. Therefore, the leaked aerosol generating substrate is guided by the ventilation channel 310, the aerosol generating substrate is guided into the installation groove 320 through one end, close to the liquid storage tank 100, of the ventilation channel 310, and is stored in the installation groove 320, so that reduction of the ventilation efficiency of the aerosol generating apparatus is avoided, and the core of the aerosol generating apparatus is prevented from being burnt.

In the prior art, a Tesla valve structure or a labyrinth structure is used for the ventilation channel 310, so that the leakage of the aerosol generating substrate into the mainboard to damage the main machine can be avoided. However, due to accumulation of the aerosol generating substrate within the ventilation channel 310, the aerosol generating substrate accumulated in the ventilation channel 310 is pushed back into the liquid storage tank 100 by atmospheric pressure during the ventilation process of the aerosol generating apparatus, so that the ventilation efficiency is reduced, resulting in a defective aerosol generating apparatus with a burnt core.

According to the present disclosure, with a the ventilation channel 310 having gradually increased diameter from an end in communication with the installation groove 320 toward the liquid storage tank 100, (that is, the ventilation channel is of a structure with a bigger top and a smaller bottom), the leakage of the aerosol generating substrate into the ventilation channel 310 can be rapidly guided into the installation groove 320, so that the aerosol generating substrate is stored in the installation groove 320. Thus, accumulation of the aerosol generating substrate in the ventilation channel 310 and negative impact on the smooth operation of the aerosol generating apparatus during ventilation can be avoided, the leakage of the aerosol generating substrate into a mainboard to damage the aerosol generating apparatus is prevented, the ventilation efficiency of the aerosol generating apparatus is improved, and a defective product with a burnt core is avoided.

In an embodiment, referring to FIG. 1 to FIG. 6, the ventilation channel 310 includes a first channel 311 and a second channel 312. The liquid storage tank is in communication with the installation groove 320 through the first channel 311, and the first channel 311 is in communication with the atomization chamber 200 through the second channel 312. Specifically, the ventilation channel 310 includes a first channel 311 and a second channel 312. The first channel 311 is a main channel of the ventilation channel 310, and the installation groove 320 is in communication with the liquid storage tank 100 through the first channel 311. The first channel 311 is gradually increased in diameter toward the liquid storage tank 100 from an end in communication with the installation groove 320. The first channel 311 plays a role of quick guidance. In a case where a part of the aerosol generating substrate leaks out of the liquid storage tank 100, the aerosol generating substrate is guided by the first channel 311 to flow into the installation groove 320 and is adsorbed by an absorber 400 located in the installation groove 320. The second channel 312 is provided along the first channel 311. One end of the second channel 312 is in communication with the first channel 311, and another end of the second channel 312 is in communication with the atomization chamber 200. The position where the second channel 312 is in communication with the first channel 311 is located above the installation groove 320. Therefore, the second channel 312 serves as an inflow channel for airflow. Air flows from the atomization chamber 200 into the first channel 311 through the second channel 312, thus accumulation of excessive aerosol generating substrate in the first channel 311 and the second channel 312 and reduction of the ventilation efficiency are avoided.

In a specific embodiment, referring to FIG. 1 to FIG. 6, the second channel 312 includes a first connecting section 3121, a bending section 3122, and a second connecting section 3123. One end of the first connecting section 3121 is in communication with the first channel 311. The first connecting section 3121 extends from an end in communication with the first channel 311 along a direction close to the liquid storage tank 100 and away from the first channel 311 to be connected with the bending section 3122. Another end of the bending section 3122 is connected with the second connecting section 3123. Another end of the second connecting section 3123 is in communication with the atomization chamber 200. Specifically, the second channel 312 includes a first connecting section 3121, a bending section 3122, and a second connecting section 3123. The first connecting section 3121 is in communication with the first channel 311. The first connecting section 3121 extends from an end in communication with the first channel 311 along a direction close to the liquid storage tank 100 and away from the first channel 311, that is, the first connecting section 3121 extends obliquely upward, so that a part of the aerosol generating substrate accumulated in the first connecting section 3121 can be guided by the first channel 311 into the first channel 311 and finally into the installation groove 320. Due to the fact that the atomization chamber 200 is located below the liquid storage tank 100, and one end of the second connecting section 3123 is required to be in communicate with the atomization chamber 200, the second connecting section 3123 is formed by extending from one end in communication with the atomization chamber 200 along a direction toward the liquid storage tank 100 and close to the first channel 311. Thus, the extension direction of the second connecting section 3123 is opposite to the extension direction of the first connecting section 3121, that is, the second connecting section 3123 extends obliquely downward. A bending section 3122 is formed in the second channel 312. The first connecting section 3121 is connected with the second connecting section 3123 through the bending section 3122. That is, one end of the bending section 3122 is connected with one end of the first connecting section 3121, and another end of the bending section 3122 is connected with one end of the second connecting section 3123. Therefore, in the course of air flowing from the second connecting section 3123 to the first connecting section 3121, the flow rate remains constant, and a part of the aerosol generating substrate is not easily accumulated in the second channel 312, thus reduction of the ventilation efficiency is avoided. With the bending section 3122, the structure of the second channel 312 is stable, and the second channel 312 is not easily damaged.

In an embodiment, referring to FIG. 1 to FIG. 6, an angle α between the first connecting section 3121 and the first channel 311 in a direction toward the liquid storage tank 100 is greater than 30 degrees. Specifically, the first connecting section 3121 is in communication with the first channel 311 and the first connecting section 3121 is formed by extending obliquely upward, so that an angle is formed between the first connecting section 3121 and the first channel 311, and the angle is directed toward the liquid storage tank 100. The angle is set to be 30 degrees or more, so that the flow rate can be kept constant when the air flows, avoiding a series of problems such as noise caused by a too fast flow rate, and facilitating the arrangement of the first connecting section 3121. In particular, in this embodiment, the angle is set to be 37 degrees, which allows a part of the leaked aerosol generating substrate to flow into the first channel 311 more rapidly, and also allows the air to flow smoothly into the first channel 311, thereby ensuring the structural stability of the ventilation channel 310.

In a specific embodiment, as shown in FIG. 1 to FIG. 6, an outer wall of the supporting frame 300 is recessed inwardly to form the ventilation channel 310, and the ventilation channel does not penetrate through the supporting frame 300. Specifically, the outer wall of the supporting frame 300 refers to a wall surface on an outer side of the supporting frame 300, that is, the outer wall of the supporting frame 300 faces the outside of the aerosol generating apparatus. The outer wall of the supporting frame 300 is recessed inwardly to form the ventilation channel 310, and the ventilation channel does not penetrate through both inner and outer sides of the supporting frame 300. That is, the ventilation channel 310 is formed as a groove. The ventilation channel 310 is integrated with the supporting frame 300, which can reduce production costs, simplify production, and facilitate installation.

In an embodiment, as shown in FIG. 1 to FIG. 6, the outer wall of the supporting frame 300 is recessed inwardly to form the installation groove 320, and both ends of the installation groove 320 extend through both sides of the supporting frame 300 in a direction perpendicular to the first channel 311. Specifically, the outer wall of the supporting frame 300 is recessed inwardly to form the installation groove 320, and the installation groove 320 is located below the first channel 311. The installation groove 320 is in communication with the first channel 311. Both ends of the installation groove 320 extend through both sides of the supporting frame 300 in a direction perpendicular to the first channel 311. That is, the left and right sides of the installation groove 320 extend through two opposite sides of the supporting frame 300. Thus, the installation groove 320 is in the form of a notch in the direction facing the outside of the supporting frame 300, which facilitates the installation of the absorber 400 in the installation groove 320, resulting in low production costs and easy assembly.

In a specific embodiment, as shown in FIG. 1 to FIG. 6, the depth of the installation groove 320 recessed is greater than the depth of the ventilation channel 310 recessed. Specifically, the depth of the installation groove 320 recessed inwardly from the outer wall of the supporting frame 300 is greater than the depth of the ventilation channel 310 recessed inwardly from the outer wall of the supporting frame 300, which facilitates the arrangement of the absorber 400. Moreover, the volume of the installation groove 320 is greater than the volume of the ventilation channel 310, so that when the aerosol generating substrate is guided to the installation groove 320, a greater amount of aerosol generating substrate will be stored.

In an embodiment, referring to FIG. 1 to FIG. 6, the ventilation channel 310 includes one first channel 311 and two second channels 312, the two second channels 312 are located on both sides of the first channel 311. Specifically, the ventilation channel 310 includes one first channel 311 and two second channels 312, and the two second channels 312 are arranged along the height direction of the first channel 311. The two second channels 312 are arranged on both sides of the first channel 311, that is, one of the two second channels 312 is arranged on the left side of the first channel 311, and the other is arranged on the right side of the first channel 311. The position where one second channel 312 is in communication with the first channel 311 is arranged opposite to the position where the other second channel 312 is in communication with the first channel 311, so that air flows to the first channel 311 from the left and right sides of the first channel 311 through the two second channels 312. The arrangement of two second channels 312 can further improve the ventilation efficiency of the aerosol generating apparatus. Moreover, air flows into the first channel 311 from both sides of the first channel 311, so that the air pressure inside the first channel 311 is kept constant. Thus, the aerosol generating substrate flows smoothly to the installation groove 320, and the structural stability of the ventilation channel 310 is improved.

In a specific embodiment, as shown in FIG. 1 to FIG. 6, two ventilation channels 310 and two installation grooves 320 are formed in the supporting frame 300, the two ventilation channels 310 and the two installation grooves 320 are located on both sides of the supporting frame 300. Specifically, the ventilation channels 310 and the installation grooves 320 are formed on left and right sides of the supporting frame 300. The two ventilation channels 310 and the two installation grooves 320 are symmetrically arranged to enhance the stability of the structure. The supporting frame 300 includes a left side wall 331, a right side wall 332, a front wall 333, and a rear wall 334. The first channels 311 of the two ventilation channels 310 are formed on the left side wall 331 and the right side wall 332 of the supporting frame 300, and the second channels 312 thereof are formed on the front wall 333 or the rear wall 334 of the supporting frame 300 and in communication with the first channels 311. The front wall 333, the rear wall 334, and the left side wall 331 or the right side wall 332 of the supporting frame 300 are recessed toward the inside of the supporting frame 300 to form the installation grooves 320. The first channels 311 are in communication with the installation grooves 320. Therefore, the aerosol generating apparatus includes two ventilation structures, which can improve the ventilation efficiency of the aerosol generating apparatus. Furthermore, in a case where one ventilation structure is damaged, the other ventilation structure can ensure the normal ventilation function of the aerosol generating apparatus, so that the structural stability of the aerosol generating apparatus is improved. Also, the two ventilation channels 310 and the two installation grooves 320 are located on both sides of the supporting frame 300, which would not affect normal operation of other parts of the aerosol generating apparatus.

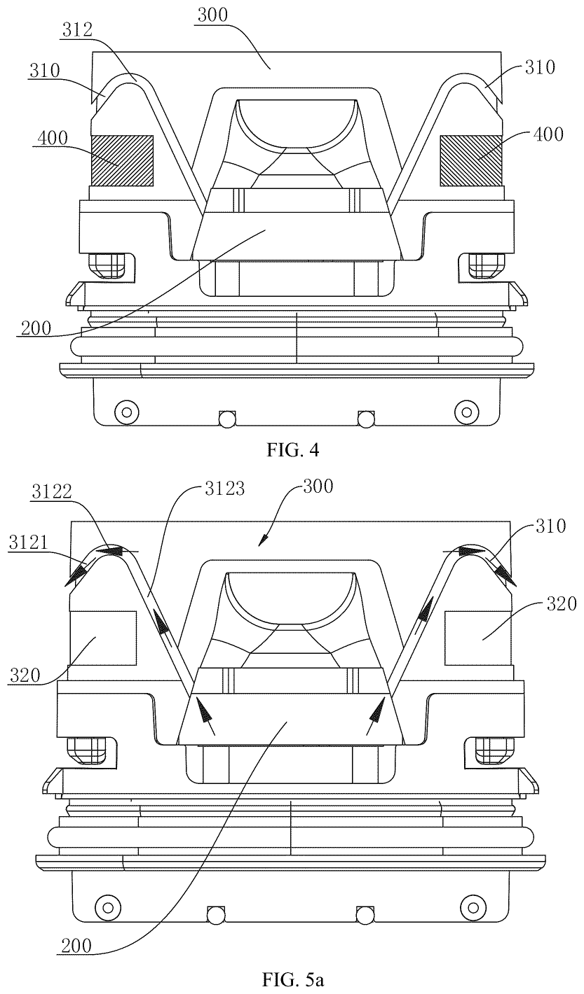

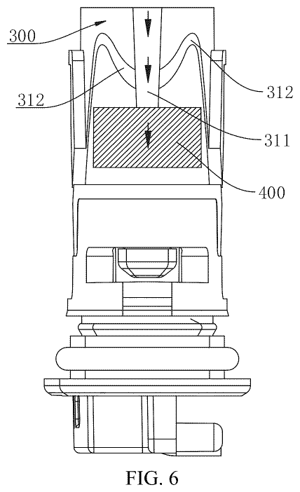

In an embodiment, referring to FIG. 4 and FIG. 6, the ventilation structure further includes an absorber 400, the absorber 400 is arranged within the installation groove 320, and the absorber 400 is configured for storing the aerosol generating substrate. Specifically, an absorber 400 is arranged in the installation groove 320. The absorber 400 is matched in size with the installation groove 320, so that the structural stability of the ventilation structure is improved. The absorber 400 is configured to absorb and store the aerosol generating substrate. The absorber 400 may be made of an adsorbent material such as oil absorbing cotton, which is not limited thereto. The absorber 400 is arranged in the installation groove 320, so that absorption of the leaked aerosol generating substrate by the ventilation structure is improved, and leakage of the aerosol generating substrate can be further avoided.

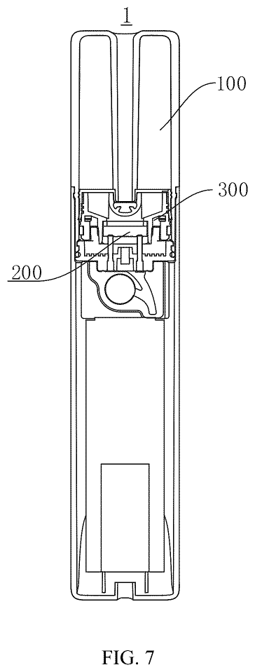

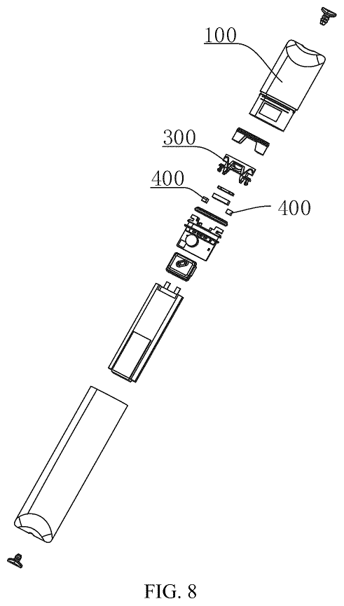

Referring to FIG. 7 and FIG. 8, this embodiment further provides an aerosol generating apparatus including a ventilation structure, any of the ventilation structures provided in the present disclosure can be used as the ventilation structure. Since the specific structure and the working principle of the ventilation structure have been described in detail in the foregoing description, details will be omitted herein for the sake of simplicity.

By adopting the ventilation structure provided in the present disclosure, the aerosol generating apparatus of the subject embodiment can avoid the aerosol generating substrate inside the aerosol generating apparatus from leaking, as well as improving ventilation efficiency of the aerosol generating apparatus, thereby avoiding undesirable phenomena such as a sticky core.

The above description is only specific embodiments of the present disclosure, and the scope of protection of the present disclosure is not limited thereto. Any equivalent modifications or substitutions may readily occur to those skilled in the art within the technical scope of the present disclosure, and such modifications or substitutions are intended to be included within the protection scope of the present disclosure. The protection scope of the present disclosure shall be subject to the claims.

What is claimed is:

1. A ventilation structure, comprising:
a liquid storage tank;
an atomization chamber located on one side of the liquid storage tank; and
a supporting frame fixedly connected to the liquid storage tank, a ventilation channel and an installation groove being formed in the supporting frame, the liquid storage tank being in communication with the installation groove and the atomization chamber through the ventilation channel, and the ventilation channel being gradually increased in diameter from an end in communication with the installation groove toward the liquid storage tank.

2. The ventilation structure according to claim 1, wherein the ventilation channel comprises a first channel and a second channel, the liquid storage tank is in communication with the installation groove through the first channel, the first channel is in communication with the atomization chamber through the second channel.

3. The ventilation structure according to claim 2, wherein the second channel comprises a first connecting section, a bending section, and a second connecting section, one end of the first connecting section is in communication with the first channel, the first connecting section extends from an end in communication with the first channel along a direction close to the liquid storage tank and away from the first channel to be connected with the bending section, another end of the bending section is connected with the second connecting section, and another end of the second connecting section is in communication with the atomization chamber.

4. The ventilation structure according to claim 3, wherein an angle between the first connecting section and the first channel in a direction toward the liquid storage tank is greater than 30 degrees.

5. The ventilation structure according to claim 3, wherein the second connecting section is formed by extending from one end in communication with the atomization chamber along a direction toward the liquid storage tank and close to the first channel.

6. The ventilation structure according to claim 2, wherein an outer wall of the supporting frame is recessed inwardly to form the ventilation channel, and the ventilation channel does not penetrate through the supporting frame.

7. The ventilation structure according to claim 6, wherein the outer wall of the supporting frame is recessed inwardly to form the installation groove, and both ends of the installation groove extend through both sides of the supporting frame in a direction perpendicular to the first channel.

8. The ventilation structure according to claim 7, wherein a depth of the installation groove recessed is greater than a depth of the ventilation channel recessed.

9. The ventilation structure according to claim 7, wherein a volume of the installation groove is greater than a volume of the ventilation channel.

10. The ventilation structure according to claim 2, wherein the ventilation channel includes one first channel and two second channels, the two second channels are located on both sides of the first channel.

11. The ventilation structure according to claim 1, wherein the supporting frame comprises a left side wall, a right side wall, a front wall and a rear wall.

12. The ventilation structure according to claim 11, wherein two ventilation channels and two installation grooves are formed in the supporting frame, first channels of the two ventilation channels are located on the left side wall and the right side wall of the supporting frame, and second channels of the two ventilation channels are located on the front wall or the rear wall of the supporting frame and in communication with the first channels.

13. The ventilation structure according to claim 12, wherein the front wall, the rear wall, and the left side wall of the supporting frame are inwardly recessed toward an inside of the supporting frame to form one installation groove of the two installation grooves, and the front wall, the rear wall, and the right side wall of the supporting frame are inwardly recessed toward the inside of the supporting frame to form another installation groove of the two installation grooves.

14. The ventilation structure of claim 1, further comprising an absorber arranged in the installation groove, and the absorber is configured for storing an aerosol generating substrate.

15. An aerosol generating apparatus, comprising:
a ventilation structure, comprising:
a liquid storage tank;
an atomization chamber located on one side of the liquid storage tank; and
a supporting frame fixedly connected to the liquid storage tank, a ventilation channel and an installation groove being formed in the supporting frame, the liquid storage tank being in communication with the installation groove and the atomization chamber through the ventilation channel, and the ventilation channel being gradually increased in diameter from an end in communication with the installation groove toward the liquid storage tank.

16. The aerosol generating apparatus according to claim 15, wherein the ventilation channel comprises a first channel and a second channel, the liquid storage tank is in communication with the installation groove through the first channel, the first channel is in communication with the atomization chamber through the second channel.

17. The aerosol generating apparatus according to claim 16, wherein the second channel comprises a first connecting section, a bending section, and a second connecting section, one end of the first connecting section is in communication with the first channel, the first connecting section extends from an end in communication with the first channel along a direction close to the liquid storage tank and away from the first channel to be connected with the bending section, another end of the bending section is connected with the second connecting section, and another end of the second connecting section is in communication with the atomization chamber.

18. The aerosol generating apparatus according to claim 17, wherein an angle between the first connecting section and the first channel in a direction toward the liquid storage tank is greater than 30 degrees.

19. The aerosol generating apparatus according to claim 16, wherein an outer wall of the supporting frame is recessed inwardly to form the ventilation channel, and the ventilation channel does not penetrate through the supporting frame.

20. The aerosol generating apparatus according to claim 19, wherein the outer wall of the supporting frame is recessed inwardly to form the installation groove, and both ends of the installation groove extend through both sides of the supporting frame in a direction perpendicular to the first channel.
